(19) United States
(12) Patent Application Publication
(10) Pub. No.: US 2026/0033777 A1
(43) Pub. Date: Feb. 5, 2026

(54) SYSTEM AND METHOD FOR THERMAL IMAGING AND SCREENING

(71) Applicant: Jordan Francis Goins, Woodstock, GA (US)

(72) Inventor: Jordan Francis Goins, Woodstock, GA (US)

(21) Appl. No.: 19/281,094

(22) Filed: Jul. 25, 2025

Related U.S. Application Data
(60) Provisional application No. 63/677,780, filed on Jul. 31, 2024.

(51) Int. Cl. A61B 5/00; A61B 5/01; G06T 7/00; G06V 10/14; G06V 10/40; G06V 10/764; G06V 10/82; G06V 40/10

(52) U.S. Cl. CPC A61B 5/4818; A61B 5/015; A61B 5/742; G06T 7/0012; G06V 10/14; G06V 10/40; G06V 10/764; G06V 10/82; G06V 40/10; G06T 2207/10048; G06T 2207/20084; G06T 2207/30196; G06V 2201/03

(57) ABSTRACT

A system and method for diagnosing sleep apnea in a subject using one or more thermal imaging cameras and a trained artificial intelligence model. Thermal images of at least a subject's facial, chest, and abdominal regions during sleep are captured for predefined duration and pre-defined intervals. The captured images are processed using an image recognition module to extract features, which are then classified using a trained artificial intelligence model to determine the likelihood of sleep apnea. The system is designed for both residential and clinical settings, enabling efficient, contactless, and user-friendly screening. The results can be presented via an application interface accessible on user devices.

### CROSS-REFERENCE TO RELATED APPLICATIONS

This application claims priority from a U.S. Provisional Patent Appl. No. 63/677,780, filed on Jul. 31, 2024, which is incorporated herein by reference in its entirety.

### FIELD OF INVENTION

The present invention relates to a system and method for thermal imaging and screening, and more particularly, the present invention relates to a system and method for thermal imaging and screening for diagnosing sleep apnea.

### BACKGROUND

Sleep apnea is a prevalent sleep disorder affecting a significant portion of the population across all age groups. Various diagnostic methods are employed to detect sleep apnea, the most established of which is polysomnography, including nocturnal polysomnography. This comprehensive diagnostic technique monitors multiple physiological parameters during sleep, such as brain wave activity via electroencephalography (EEG), eye movements via electrooculography (EOG), muscle activity and tone via electromyography (EMG), and heart rate and rhythm via electrocardiography (ECG).

Polysomnography is typically conducted overnight in a clinical sleep laboratory, where trained technicians supervise data collection to ensure accuracy. Sleep specialists then analyze the collected data to confirm a diagnosis and recommend appropriate treatment. Advancements in technology have also introduced home sleep apnea tests (HSATs), offering a more convenient and accessible alternative for some patients.

Sleep apnea thermal imaging has recently emerged as a novel, non-invasive method for screening and diagnosing sleep apnea. This approach offers significant advantages over traditional method being more convenient and comfortable to the patient. However, there is a large scope of improvement in the efficiency and accuracy of such method in diagnosing sleep apneas is still limited.

A need is therefore appreciated for advancements in sleep apnea thermal imaging to make the diagnosis safe and more convenient.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a system and method for thermal imaging and screening for diagnosing sleep apnea disorders that are safer and more convenient.

Another object of the present invention is that the method can be performed easily at home. For example, the system may use a single camera or multiple cameras positioned strategically in the room to monitor key areas such as the patient's face, chest, and abdomen. The thermal images captured by these cameras are processed by the system's AI model, which classifies the extracted features into typical or atypical for sleep apnea, providing the patient with results in real-time. This home-use capability eliminates the need for hospital visits, reducing patient exposure to hospital environments while still delivering accurate diagnostic results.

Still, another object of the present invention is that the results can be obtained quickly.

Yet another object of the present invention is that the need for hospital or clinic visit can be avoided.

An additional object of the present invention is that the system and method can make the diagnosis of sleep apnea disorders in hospital and clinical settings in a quick, efficient, and patient friendly manner

In one aspect, a system for diagnosing sleep apnea in a subject is disclosed, the system comprises one or more thermal imaging cameras configured to capture thermal images of the subject during sleep; a processor operably coupled to the one or more thermal imaging cameras; and a memory operably coupled to the processor and comprises a set of instructions, wherein the set of instructions, when executed by the processor, cause the system to receive time-stamped thermal image data of at least facial, chest, and abdominal regions of the subject; extract features from thermal images in the time-stamped thermal image data using an image recognition module; and classify the features using a trained artificial intelligence model into typical or atypical for sleep apnea, wherein the trained artificial intelligence model is a neural network trained on labeled thermal image data of individuals with and without sleep apnea. The one or more thermal imaging cameras comprises a plurality of thermal imaging cameras configured to be positioned at different angles relative to the subject.

In one aspect, disclosed is a method for diagnosing sleep apnea in a subject, the method comprising capturing thermal images of the subject during sleep, continuously for a predetermined duration, at predefined intervals, using one or more thermal imaging cameras, wherein the thermal images capture at least facial, chest, and abdominal regions of the subject, wherein the thermal images are time-stamped; generating a time-series image data from the thermal inches; extracting features from the thermal images in the time-series image data, wherein the features are based on changing patterns in the thermal images recognized using an image recognition algorithm; classifying the extracted features using an artificial intelligence (AI) model to determine if the features are typical or atypical for sleep apnea, wherein the artificial intelligence (AI) model is a neural network trained on labeled thermal image data of individuals with and without sleep apnea; and generating, a diagnosis report based on the classification. The method further comprises rendering an interface on a user device; and presenting the diagnosis report through the interface on the user device. The features extracted from the thermal images comprises at least one of body movement, breathing patterns, and thermal variations associated with respiratory events.

### BRIEF DESCRIPTION OF DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a block diagram showing the environment of the system, according to an exemplary embodiment of the present invention.

FIG. 2 is a block diagram showing the architecture of the system, according to an exemplary embodiment of the present invention.

FIG. 3 is a flowchart illustrating the method for diagnosing sleep apnea, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

The invention described pertains to a system and method for thermal imaging and screening for the diagnosis of sleep apnea. The disclosed system and method can make the diagnosis of sleep apnea safer, more convenient, efficient, reliable, and faster yet effective. The disclosed system may use the techniques of artificial intelligence for screening, making the process quicker and cost-effective. The system allows for imaging from home, preventing exposure of a person to the hospital environment. In residential settings, the system is designed to allow for easy setup and use by patients without the need for professional installation. The thermal cameras can be positioned at convenient angles around the patient, such as near the bedside or in a corner of the room, to capture the necessary thermal images. The placement of the cameras is flexible, ensuring that a patient can easily position the cameras without requiring complex equipment or technical expertise. This user-friendly design makes the system ideal for self-diagnosis, allowing patients to perform the screening in the comfort of their own home.

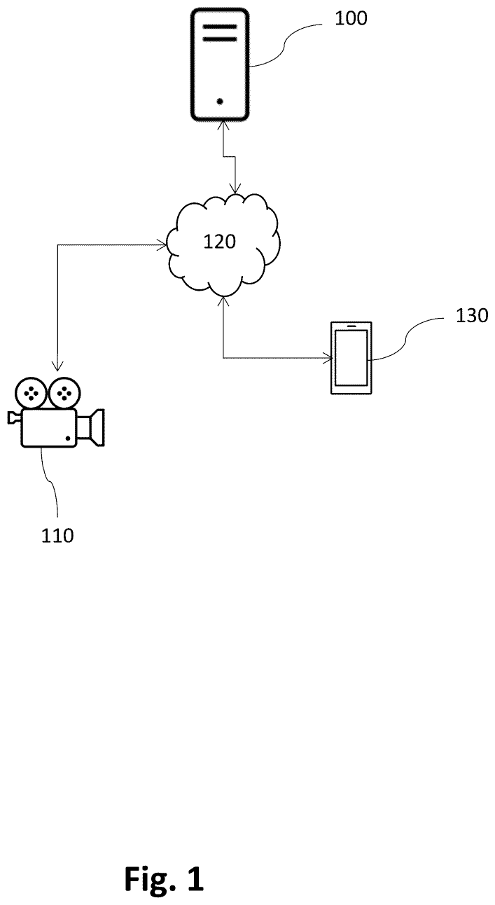

Referring to FIG. 1 which shows the environment of the disclosed system 100. System 100 can connect to one or more thermal imaging cameras 110 through a network 120. Preferably, the system can connect to multiple thermal imaging cameras. The network can be a communication network known in the art which can be a wired network, a wireless network, or may include a combination of wired and wireless networks. Examples of communication networks may be a local area network (LAN), a wide area network (WAN), a wireless WAN, a wireless LAN (WLAN), a metropolitan area network (MAN), a wireless MAN network, a cellular data network, a cellular voice network, the Internet, etc.

The system 100 may also connect to one or more user device 130 through the network. While, for illustration herein, FIG. 1 shows a single network connecting multiple user devices, it should be obvious to those reading this disclosure that different user devices can connect with the system through various networks, and the same user device can connect with the system through more than two networks. For example, a user device can connect to the system through a LAN and the Internet. The term “user” as used herein, and throughout this disclosure, refers to an individual engaging a user device to interact with the system. The user can be a person undergoing diagnostics and/or a healthcare professional. For example, the user using the user device can perform a diagnosis by themself and may optionally share the results with a healthcare professional. Also, a healthcare professional, such as a physician can use the user device for diagnosing sleep apnea. The user can perform the diagnosis at the user site or remotely, wherein the user device connects through the network.

The user device can be any computing device that includes a processor for processing instructions stored in memory. The user device can also include an input module for receiving input from the user. Such input can be in the form of a touch display, mouse, stylus, keyboard, touchpad, and the like. The user device may also include a display for presenting information to the user, such as an LCD screen. The user device may also include a network circuitry for connecting to the network 120. Examples of the user device include a smartphone, a desktop computer, a laptop, a workstation, and the like.

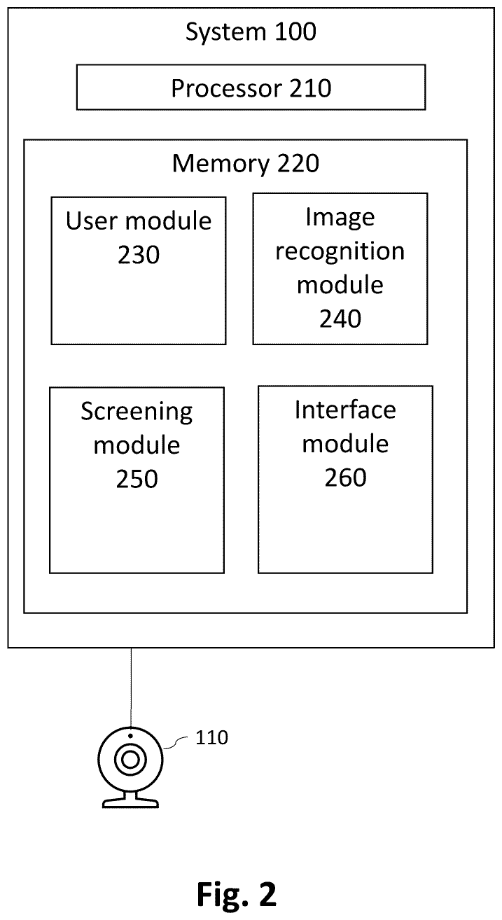

Referring to FIG. 2 is a block diagram showing the architecture of system 100. The System 100 includes a processor 210 and a memory 220 operably coupled to the processor. The processor can be any logic circuitry that responds to, and processes instructions fetched from the memory. Examples of such processors include the processors by Intel® and AMD®. Also, the processor may be suitable for powering AI models. Examples of such processors include Nvidia® Jetson Nano which may be used to power the AI workloads during the development phases. More power processors, such as Nvidia® Jetson AGX Xavier or Jetson Orin Series may be used for later development stages. The memory may include one or more memory chips capable of storing data and allowing any storage location to be directly accessed by the processor. The memory can include modules according to the present invention for execution by the processor to perform one or more steps of the disclosed methodology. The system may also include an operating system, such as Microsoft® Windows® and Linux® The system may also include one or more thermal cameras.

The system can be implemented in the form of servers, which include cloud servers. The servers can be placed in one location or geographically dispersed. Also, one or more steps of the disclosed methodology can be performed on one or more user devices without departing from the spirit of the disclosed subject matter.

The memory may include a user module 230, an image recognition module 240, a screening module 250, and an interface module 260. The user module 230, upon execution by the processor, allows for managing user registration and profile management. The image recognition module 240, upon execution by the processor, allows for processing the thermal images received from one or more cameras. The screening module 250, upon execution by the processor allows screening of the image data for diagnosing sleep apnea. The interface module upon execution by the processor provides an interface on the user device for interacting with the disclosed system 100.

The term module as used herein and throughout this disclosure refers to software, a program code, a set of rules or instructions, and the like in one or more computer-readable languages including graphics, which upon execution by the processor performs one or more steps of the disclosed methodology. Also, operations may be described as a sequential process, some of the operations may be performed in parallel, concurrently, and/or in a distributed environment, and with program code stored locally or remotely for access by single or multi-processor machines. In addition, in some implementations, the order of operations may be rearranged without departing from the spirit of the disclosed subject matter.

The interface provided by the interface module allows a user to interact with the disclosed system through a user device. The interface may include a series of screens, which in continuation can provide information as well as receive information from the user and execute one or more steps of the disclosed methodology. The interface can be dynamic and allows switching between sections, screens, pages, and the like quickly and easily. The interface can be provided as an application software that can be installed on the user device. The application software can be developed for Android™, iOS, and any other known operating platform for mobile devices. The application software can be made available through a distribution service provider, for example, Google Play™ operated and developed by Google, and the app store by Apple. In addition to the application software, a website-based interface can also be provided through the World Wide Web. The application software can also be provided for the desktop environment, such as Windows™, Linux, and macOS. The user interface may permit interaction with a user through the user device, wherein information can be presented within the user interface by system 100 and information can be received by system 100 from the user.

The user module may allow an individual willing to use the disclosed system to register. The user module can receive basic information about the individual, such as name, contact details, email address, and the like. The user module can generate a profile for the user and store the same in a suitable database. The databases, including their structure and functioning, are known in the art. Also, the use of blockchain databases is well known. The present invention can use any suitable database without departing from the scope of the present invention. Also, the user module may allow the profile created to be later modified by the user. The information can be received through questionaries, wizards, and the like. For example, the user may be asked questions about their health, medical condition, sleeping patters, eating patterns, and the like.

The profile can include all such information about the user including age, gender, weight, and the like. The information, such as sleeping time and duration can be obtained from the user. The system may also connect with external devices and databases to obtain information about the user. Examples of external devices include activity tracking wrist bands. Examples of external databases using electronic medical records.

The user module can generate login details to access the disclosed system securely. The login details may include at least a username and a password. The password can be an alphanumeric code, or biometric like a fingerprint, token, and the like. The user may have multiple login options, such as using an alphanumeric code or a fingerprint. Also, the use of multiple-factor authentication is within the scope of the present invention. The user can be provided with a login screen on the user device for accessing the disclosed system.

First, a thermal camera may be positioned to capture images of a person sleeping to record the breathing. The camera may focus at least on the abdomen, chest, and face of the person. Since the body naturally repositions while sleeping, it may be preferable to use more than one camera. Recording by the cameras may be time-stamped to obtain time-series-image data. The person can be a baby, a child, or an adult. The present invention is particularly useful for children and infants, as the invention does not require any application of wires or devices to the body. The person or patient being subjected to the disclosed system or wish to use the disclosed system for diagnosing sleep apnea is also referred to herein as a subject.

The time series image data may be received by the image recognition module 240. The image recognition module 240 can recognize patterns in the images by using various known image recognition algorithms. The image recognition module 240 can dissect the images to extract relevant features. Also, the redundancy of the obtained image data can be reduced by the image recognition module 240. The image recognition module 240 may be based on Convolutional Neural Networks (CNNs).

The features extracted by the image recognition module 240 can be received by the screening module 250. The screening module 250 uses a trained AI model to distinguish features. The AI model may be trained using thermal images of people suffering from sleep apnea and people not suffering from sleep apnea. The screening module can visualize the breathing patterns from thermal images captured from one or more cameras. The classifier of the AI model can classify the features extracted from thermal images into typical or atypical for sleep apnea. Typically, Convolutional Neural Networks (CNNs) trained on single-channel grayscale data (since thermal images are not RGB) may be used. Pooling of the feed from multiple cameras, capturing multiple angles for the same subject, may allow for better diagnosis by allowing for focusing on regions of high heat contrast (e.g., face, chest, hands) and makes the model more robust to angle changes. For pooling, multi-View CNN can be used that can process the feed from multiple angles for the same subject into parallel CNN streams (same weights). Concatenate the feature vectors and classify using a final dense layer.

Use of thermal imaging from multiple angles of the same subject offers several advantages. Multiple angles allow the creation of a more complete thermal profile of the subject. This enables better 3D reconstruction, which helps image recognition understand shape, posture, and spatial relationships. It reduces the risk of errors caused by occlusions (e.g., when parts of the body are hidden in one view but visible in another). Capturing multiple perspectives helps the system become pose-invariant, meaning it can recognize the person regardless of how they are facing. Also, a richer and diverse data set can be generated for diagnosis and training. In security or medical settings, multi-angle imaging can provide more reliable biometric data, like thermal patterns of the face or vascular features. This reduces false positives and false negatives in identification tasks

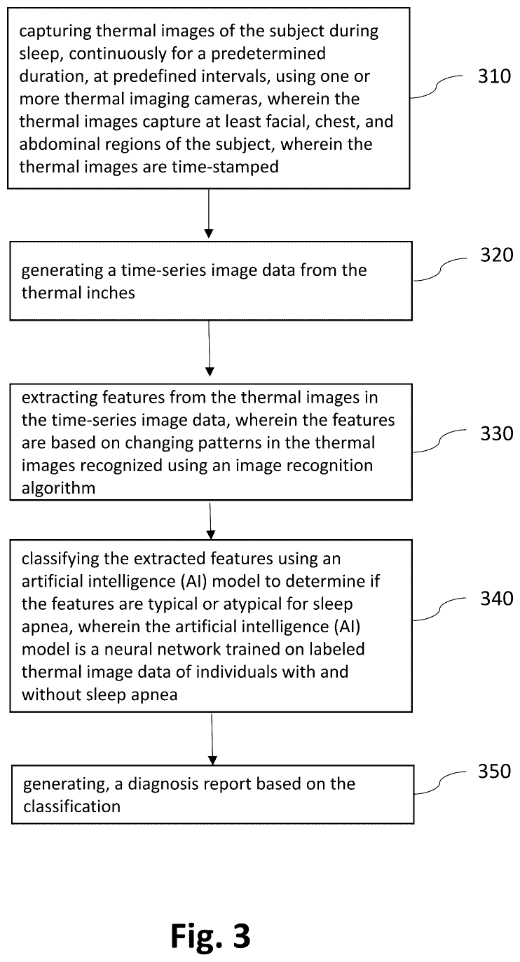

Referring to FIG. 3 which is a flowchart illustrating the method for diagnosing sleep apnea in a subject. First, thermal images of the subject during sleep can be captured continuously for a predetermined duration, at predefined intervals, using one or more thermal imaging cameras, at step 310. For example, an image can be captured at an interval of 10 sec for 2 hours. The thermal images cover at least facial, chest, and abdominal regions of the subject. Also, the thermal images can be time-stamped. A time-series image data from thermal images can be created by the system, at step 320. The images can be pre-processed. To standardize the images, the images can be converted to standard formats (e.g., PNG, JPEG, or NumPy arrays) with preserved temperature information. The standardized images may be of the same size, quality and coloration. Also, all images may use the same coloration.

This thermal image data can be analysis in near real time or later, offering flexibility and versatility to a subject. The system can extract features from the thermal images, wherein the features are based on changing patterns in the thermal images recognized using an image recognition algorithm, at step 330. The features may include changing patterns in the time-series image data as recognized by image recognition algorithm. The features include body movement, breathing patterns, and thermal variations associated with respiratory events. Upon extracting the features for the image data, an artificial intelligence (AI) model classifies the extracted features into typical or atypical for sleep apnea, at step 340. The artificial intelligence (AI) model is a neural network trained on labeled thermal image data of individuals with and without sleep apnea. A diagnosis report based on the result of the classification can be generated by the system, at step 350. This report can be rendered through an interface on a user device.

Disclosed is a method for diagnosing sleep apnea in a subject. The method includes collecting thermal images of a subject from multiple angles (e.g., front, side, back). Organize the dataset of thermal images into training, validation, and test sets. The folders in these folders may be where the datasets are separated into the severity of sleep apnea the images have. New images can be categorized using these old images. The directories can be structured by individual IDs and angles. The thermal images are to be converted to single-channel grayscale (if not already). Also, the pre-processing step of images includes resizing and normalizing pixel values (e.g., 0-255 scaled to 0.0-1.0). Optionally augmenting the image data using rotation, flipping, and noise to improve generalization.

Convolutional Neural Networks (CNNs) can be used for thermal image feature extraction. Multi-View CNN can be used for multiple angle input. For multiple angle feed includes, feeding each angle into a separate CNN stream with shared weights. Each CNN of the Multi-View CNN includes convolution layers followed by pooling layers to reduce dimensionality and focus on important thermal regions. Implementing pooling (e.g., MaxPooling or Global Average Pooling) to emphasize high-contrast heat regions like the face, chest, and hands, and improve robustness to shifts or noise. Thereafter, concatenate the feature vectors from each CNN stream. Passing the concatenated features through one or more fully connected (dense) layers.

The CNN may include an output layer. A softmax layer can be used for classification by identity. Or triplet loss or contrastive loss can be used for similarity-based recognition.

The model can be trained using the training set. Thereafter, the model can be validated using the validation set to adjust hyperparameters and prevent overfitting. Metrics, such as accuracy, loss, and F1-score may be monitored during training. Final model performance on the test set using unseen individuals or new angle combinations can then be evaluated. This trained and validated model can be used for the diagnosis of sleep apnea.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A system for diagnosing sleep apnea in a subject, the system comprises:
one or more thermal imaging cameras configured to capture thermal images of the subject during sleep;
a processor operably coupled to the one or more thermal imaging cameras; and
a memory operably coupled to the processor and comprises a set of instructions, wherein the set of instructions, when executed by the processor, cause the system to:
receive time-stamped thermal image data of at least facial, chest, and abdominal regions of the subject;
extract features from thermal images in the time-stamped thermal image data using an image recognition module; and
classify the features using a trained artificial intelligence model into typical or atypical for sleep apnea, wherein the trained artificial intelligence model is a neural network trained on labeled thermal image data of individuals with and without sleep apnea.

2. The system of claim 1, wherein the one or more thermal imaging cameras comprises a plurality of thermal imaging cameras configured to be positioned at different angles relative to the subject.

3. A method for diagnosing sleep apnea in a subject, the method comprising:
capturing thermal images of the subject during sleep, continuously for a predetermined duration, at predefined intervals, using one or more thermal imaging cameras, wherein the thermal images capture at least facial, chest, and abdominal regions of the subject, wherein the thermal images are time-stamped;
generating a time-series image data from the thermal images;
extracting features from the thermal images in the time-series image data, wherein the features are based on changing patterns in the thermal images recognized using an image recognition algorithm;
classifying the extracted features using an artificial intelligence (AI) model to determine if the features are typical or atypical for sleep apnea, wherein the artificial intelligence (AI) model is a neural network trained on labeled thermal image data of individuals with and without sleep apnea; and
generating, a diagnosis report based on the classification.

4. The method of claim 3, wherein the method further comprises:
rendering an interface on a user device; and
presenting the diagnosis report through the interface on the user device.

5. The method of claim 4, wherein the one or more thermal imaging cameras comprises a plurality of thermal imaging cameras positioned at different angles relative to the subject.

6. The method of claim 1, wherein the features extracted from the thermal images comprises at least one of body movement, breathing patterns, and thermal variations associated with respiratory events.
